(19) United States
(12) Patent Application Publication
(10) Pub. No.: US 2007/0160664 A1
(43) Pub. Date: Jul. 12, 2007

(54) Pharmaceutical compositions comprising of proton pump inhibitor and prokinetic agent

(75) Inventors: Rajesh Jain, New Delhi (IN); Kour Chand Jindal, New Delhi (IN); Sukhjeet Singh, New Delhi (IN)

Correspondence Address: LADAS & PARRY, 26 WEST 61ST STREET, NEW YORK, NY, 10023, US

(73) Assignee: PANACEA BIOTEC LIMITED

(21) Appl. No.: 11/482,186

(22) Filed: Jul. 6, 2006

Related U.S. Application Data
(63) Continuation-in-part of application No. PCT/IN05/00002, filed on Jan. 5, 2005.

(30) Foreign Application Priority Data
Jan. 6, 2004 (IN) 21/DEL/2004
Jan. 6, 2004 (IN) 25/DEL/2004

(51) Int. Cl. A61K 31/4439; A61K 9/64; A61K 9/24

(52) U.S. Cl. 424/456; 424/472; 514/338

(57) ABSTRACT

Oral pharmaceutical compositions and process for preparation thereof are provided comprising at least one gastric acid suppressing agent, preferably a proton pump inhibitor or its pharmaceutically acceptable salts, esters, hydrates, derivatives or prodrugs, and one or more prokinetic agent or its pharmaceutically acceptable salts, esters, hydrates, derivatives or prodrugs, optionally with other pharmaceutically acceptable excipients, characterized in that the gastric acid suppressing agent is present in a delayed release form and the prokinetic agent is present in a bimodal release form such as an immediate release form to provide an initial loading dose, and a delayed release form to provide a dose with a lag time; with the proviso that the prokinetic agent is not formulated using a hydrophilic rate controlling polymer and is not present in a sustained release form. Method of treatment of gastro esophageal reflux disease, reflux esophagitis, peptic ulcer, gastric ulcer, and other gastric acid related disorders by administering to a patient such a pharmaceutical composition in need thereof is also provided.

### FIELD OF THE INVENTION

The present invention relates to pharmaceutical compositions and process for preparing such compositions comprising of at least one gastric acid suppressing agent and one or more prokinetic agent(s) exhibiting a unique bimodal release profile, optionally with other pharmaceutically acceptable excipients. Preferably, the present invention describes pharmaceutical compositions of a proton pump inhibitor and one or more prokinetic agent(s). More preferably, the present invention relates to pharmaceutical composition of pantoprazole or its pharmaceutically acceptable salts, esters, hydrates, or derivatives; and domperidone or its pharmaceutically acceptable salts, esters, hydrates, or derivatives. These compositions are especially useful in the treatment of gastro esophageal reflux disease. Furthermore, the present invention refers to a method for the manufacture of such preparations in a way such that there is increased dissolution of the prokinetic agent at alkaline pH.

### BACKGROUND OF THE INVENTION

Gastro esophageal reflux disease (GERD), reflux esophagitis, peptic ulcer, gastric ulcer and other gastric acid related disorders are disorders having a pathogenesis related to reduced gastric motility and release of excessive gastric acid. Aside from behavioral changes, GERD and gastric ulcer have been successfully treated with a range of gastric acid inhibitors, such as ranitidine and omeprazole, which are acid-suppressing agents. Stimulation of gastric motility has been proposed to accelerate the healing of gastric ulcer. Prokinetic agents, such as domperidone, are known to enhance gastrointestinal motility and prevent duodenogastric reflux, and are widely used to treat GERD. Proton pump inhibitors and prokinetic agents have been used in combination to treat gastric ulcer and other related disorders.

Proton pump inhibitors, such as Lansoprazole, Omeprazole, Pantoprazole are rapidly taking share from H₂ receptor antagonists, particularly in reflux esophagitis. Omeprazole is known to offer significant gain over H₂ receptor antagonists in terms of symptom resolution, healing and prevention of relapse for reflux esophagitis.

A combination therapy of a prokinetic agent and a gastric acid lowering compound is rational and has shown more effectiveness than mono-therapy of proton pump inhibitors. Administration of cisapride and ranitidine was shown to further lower the exposure of the oesophagus to acid(s) (Inauen W et al. Gut 1993; 34: 1025-1031). Such a therapy was also shown to improve healing rates (de Boer W A et al. Aliment Pharmacol Ther 1994; 8: 147-157).

Maintenance therapy is often necessary to prevent recurrent symptoms and esophagitis. A combination therapy combining an acid-suppressing agent with a prokinetic agent has been recently described. [Vyneri et al; N. Engl. J. Med 1995; 333: 1106-1110].

U.S. Pat. No. 6,132,771 discloses a combination therapy of proton pump inhibitor and a prokinetic agent wherein, the prokinetic agent may be in the form of instant release, sustained release or extended release formulations. However, prokinetic agents such as domperidone require optimum binding to receptors. Hence, improved therapeutic efficacy may be achieved by administering the drug in timed release form with an initial loading dose and a delayed release dose provided with a lag time.

The WO Publication No. 95/01803 describes a pharmaceutical composition of famotidine, cisapride and optionally simethicone in the treatment of gastrointestinal distress.

The WO Publication No. 200471374A2 describes pharmaceutical compositions for once a day oral administration, comprising at least one delayed release component, wherein said delayed release component comprises a proton pump inhibitor, said composition further including at least one immediate release and/or a sustained release prokinetic agent. The said application discloses use of polymers to formulate sustained release compositions of the prokinetic agent. However, such compositions suffer from a major disadvantage in terms of absorption of the prokinetic agent which are primarily absorbed from the intestine and hence a delayed release composition is highly desirable.

Nagarsenker, M. S.; Garad, S. D.; Ramprakash, G., [Journal of Controlled Release (2000), 63(1-2), 31-39] describe coevaporates of domperidone prepared using different polymers by solvent evaporation technique. The drug release rate was dependent on the concentration of polymers in the coevaporates. Dissolution of drug in a pH 6.8 buffer improved with increasing concentration of hydroxypropyl methyl cellulose phthalate in coevaporates.

However, there still exists a need to develop pharmaceutical compositions comprising a combination of a gastric acid suppressing agent preferably a proton pump inhibitor and a prokinetic agent wherein the prokinetic agent is present in an immediate release form and a delayed release form useful for the treatment of gastro esophageal reflux disease, reflux esophagitis, peptic ulcer, gastric ulcer, and other gastric acid related disorders. The delayed release form of the prokinetic agent is highly essential since most of the prokinetic agents show a better absorption generally from the intestinal region of the GIT, which is an objective of the present invention.

### SUMMARY OF THE INVENTION

It is an objective of the present invention to provide an oral pharmaceutical composition comprising at least one gastric acid suppressing agent and one or more prokinetic agent, optionally with other pharmaceutically acceptable excipients, characterized in that the gastric acid suppressing agent is present in a delayed release form and the prokinetic agent is present in a bimodal release form such as an immediate release form to provide an initial loading dose, and a delayed release form to provide a dose with a lag time; with the proviso that the prokinetic agent is not formulated using a rate controlling polymer and is not present in a sustained release form.

It is an objective of the present invention to provide oral pharmaceutical composition comprising a proton pump inhibitor, preferably pantoprazole or its pharmaceutically acceptable salts, esters, hydrates, derivatives or prodrugs, and domperidone or its pharmaceutically acceptable salts, esters, hydrates, or derivatives thereof.

It is also an objective of the present invention to provide a process for preparing a composition comprising at least one gastric acid suppressing agent and one or more prokinetic agent, optionally with other pharmaceutically acceptable excipients, characterized in that the gastric acid suppressing agent is present in a delayed release form and the prokinetic agent is present in a bimodal release form such as an immediate release form to provide an initial loading dose, and a delayed release form to provide a dose with a lag time; with the proviso that the prokinetic agent is not formulated using a rate controlling polymer and is not present in a sustained release form, which comprises of the following steps:

i) processing the acid suppressing agent with pharmaceutically acceptable excipients,

ii) processing the prokinetic agent with pharmaceutically acceptable excipients,

iii) formulating the material of step i) and ii) into a suitable dosage form.

It is yet another objective to provide a method of treatment of gastro esophageal reflux disease, reflux esophagitis, peptic ulcer, gastric ulcer, and other gastric acid related disorders by administering to a patient in need thereof a pharmaceutical composition of the present invention.

### DETAILED DESCRIPTION OF INVENTION

A combination therapy comprising a gastric acid suppressing agent and a prokinetic agent is attractive, rational and effective. A combination of gastric acid suppressing agent and prokinetic agent could be an alternative to each of them separately in case of failure. However, because of the large number of therapeutical tablets/pills that must be taken each day in such a therapy, the compliance of such a treatment may be a problem. It is well known that patient compliance is a major factor in receiving good results in medical treatments. Administration of two, three or even more different tablets to the patient is not convenient or satisfactory to achieve the most optimal results. The present invention now provides new oral dosage forms comprising two or more different active substances combined in one fixed unit dosage form, preferably tablets in a capsule.

The present invention relates to pharmaceutical compositions comprising of at least one gastric acid suppressing agent and one or more prokinetic agent(s) optionally with other pharmaceutically acceptable excipients. Preferably, the present invention describes pharmaceutical compositions of a proton pump inhibitor and one or more prokinetic agent(s). More preferably, the present invention relates to pharmaceutical composition of pantoprazole or its pharmaceutically acceptable salts, esters, hydrates, derivatives or prodrugs; and domperidone or its pharmaceutically acceptable salts, esters, hydrates, derivatives or prodrugs.

One aspect of the present invention relates to oral pharmaceutical compositions of gastric motility modifying agents and their combination therapies wherein the gastric motility modifying agent has a unique bimodal release profile. The prokinetic agent is present in a bimodal release form such as an immediate release form to provide an initial loading dose, and a delayed release form to provide a dose with a lag time; with the proviso that the prokinetic agent is not formulated using a rate controlling polymer and is not present in a sustained release form. These preparations are especially useful in the treatment of gastro-esophageal reflux disease.

The proton pump inhibitor of the present invention is selected from but not limited to a group comprising pantoprazole, lansoprazole, omeprazole, esomeprazole, rabeprazole, and the like, their pharmaceutically acceptable salts, esters, hydrates, derivatives or prodrugs, used either alone or in combination thereof.

The prokinetic agent of the present invention is selected from but not limited to a group comprising domperidone, metoclopramide, itopride, mosapride, cisapride, renzapride, zacopride, octreotide, naloxone; erythromycin and bethanechol, Motilides such as Motilin, and the like, their pharmaceutically acceptable salts, esters, hydrates, derivatives or prodrugs, used either alone or in combination thereof. Preferably the prokinetic agent is domperidone, or metoclopramide, or pharmaceutically acceptable salts, esters, hydrates, or derivatives thereof.

The other pharmaceutically acceptable excipients of the present invention are selected from but not limited to the group comprising of diluents, binders, disintegrants, colorants, lubricants, plasticizers, coating agents, opacifiers, antioxidants, and the like used either alone or in combination thereof.

Suitable diluents according to the present invention are selected from but not limited to a group comprising microcrystalline cellulose such as Avicel® PH 101, Avicel® PH 102, Avicel® PH 112, Avicel® PH 200, Avicel® PH 301 and Avicel® PH 302, lactose such as lactose monohydrate, lactose anhydrous and Pharmatose® DCL 21, dibasic calcium phosphate, saccharides such as mannitol, Pearlitol® SD 200, starch, sorbitol, sucrose, and glucose; alkaline agents such as magnesium oxide, sodium bicarbonate, or mixtures thereof.

Suitable disintegrants according to the present invention are selected from but not limited to a group comprising crosslinked polyvinyl pyrrolidone, polyvinyl pyrrolidone, corn starch, potato starch, maize starch and modified starches, croscarmellose sodium, sodium starch glycollate, low substituted hydroxypropyl cellulose, or mixtures thereof.

Suitable lubricants according to the present invention are selected from but not limited to colloidal silicon dioxide, talc, stearic acid, magnesium stearate, calcium stearate, zinc stearate, and sodium stearyl fumarate, or mixtures thereof.

Suitable coating materials according to the present invention are selected from but not limited to Hydroxypropylmethyl cellulose, Eudragit L-100, Eudragit L-100 55, Opadry® yellow 03B52544 (Colorcon), Opadry® white OY-IN-58901 (Colorcon), Opadry® pink 03B54579 (Colorcon), Triethyl citrate, Propylene glycol, Colloidal silicon dioxide, Talc, Isopropyl alcohol, Dichloromethane, Purified water, and the like.

During developmental studies of the present invention, it was surprisingly found that when domperidone was co-processed with an organic acid it exhibited an improved dissolution even at alkaline pH conditions encountered in the gastro-intestinal tract.

According to a preferred embodiment of the invention, domperidone is co-processed with an organic acid in the ratio of from about 1:0.25 to about 0.25:1, preferably from about 1:0.5 to about 0.5:1, most preferably about 1:1. The co-processing may be aided by dissolving the two ingredients with the help of heat followed by cooling, when the dissolved material separates out. The material separated out may be removed and dried. The co-processed material may be incorporated into dosage forms such as tablets, which may further be combined with enteric coated tablets of proton pump inhibitor and an immediate release tablet of domperidone, in a hard gelatin capsule.

In yet another embodiment, the composition comprises the prokinetic agent as 5 to 70% by weight of total prokinetic agent in immediate release form and the remaining prokinetic agent in delayed release form.

In another embodiment of the present invention, the composition of the prokinetic agent present in immediate release form and delayed release form comprises a permeation enhancer, preferably Vitamin E tocopheryl propylene glycol succinate.

In an embodiment, the composition of the present invention is in the form of a multiparticulate composition comprising a blend of one or more types of particles, pellets or mini-tablets having different release characteristics, optionally filled into a capsule; or a tablet, or formulated as a liquid dosage form.

The present invention provides oral dosage forms, such as multiple unit tableted dosage form, or a capsule filled with more than one pharmaceutically active compound. The active compounds present in the dosage form are preferably an acid susceptible proton pump inhibitor, which is protected by an enteric coating layer, and one or more prokinetic agents. The prokinetic agent is preferably incorporated as a better dissolving complex with bimodal release. These new compositions intend to simplify the regimen and improve the patient compliance.

In a preferred embodiment of the present invention, the composition is in the form of tablets filled into hard gelatin capsule, or in the form of multilayer tablets.

In another embodiment, a process for preparing a composition is provided comprising at least one gastric acid suppressing agent and one or more prokinetic agent, optionally with other pharmaceutically acceptable excipients, characterized in that the gastric acid suppressing agent is present in a delayed release form and the prokinetic agent is present in a bimodal release form such as an immediate release form to provide an initial loading dose, and a delayed release form to provide a dose with a lag time; with the proviso that the prokinetic agent is not present in a sustained release form, which comprises of the following steps:

i) processing the acid suppressing agent with pharmaceutically acceptable excipients into enteric coated tablets,

ii) processing the prokinetic agent with pharmaceutically acceptable excipients partly into film coated tablets and partly into enteric coated tablets,

iii) filling one enteric coated tablet comprising an acid suppressing agent, and one film coated tablet and one enteric coated tablet comprising a prokinetic agent, into a hard gelatin capsule.

In a further embodiment, the process for preparing a composition is provided comprising at least one gastric acid suppressing agent and one or more prokinetic agent, optionally with other pharmaceutically acceptable excipients, characterized in that the gastric acid suppressing agent is present in a delayed release form and the prokinetic agent is present in a bimodal release form such as an immediate release form to provide an initial loading dose, and a delayed release form to provide a dose with a lag time; with the proviso that the prokinetic agent is not present in a sustained release form, which comprises of the following steps:

i) processing the acid suppressing agent with pharmaceutically acceptable excipients into enteric coated granules,

ii) processing one part of the prokinetic agent with pharmaceutically acceptable excipients into immediate release granules, and the other part into enteric coated granules,

iii) compressing the granules of step i) and ii) into a multilayer tablet,

iv) optionally coating the tablet.

The examples given below serve to illustrate embodiments of the present invention. However, they do not intend to limit the scope of the present invention.

### EXAMPLE 1

### Pantoprazole and Domperidone Tablets in a Capsule

| | |
|---|---|
| Ingredients | Quantity (mg/Tablet) |

Part A: Pantoprazole Tablets (Delayed release)

| | |
|---|---|
| Pantoprazole sodium sesquihydrate | 45.10 |
| (equivalent to Pantoprazole 40 mg) | |
| Sodium carbonate (anhydrous) | 10.00 |
| Microcrystalline cellulose | 20.90 |
| Croscarmellose sodium | 20.00 |
| Magnesium stearate | 2.00 |
| Talc | 2.00 |
| SEAL COATING FORMULA | |
| Opadry ® yellow 03B52544 | 2.0 |
| Isopropyl alcohol | q.s. |
| Dichloromethane | q.s. |
| ENTERIC COATING FORMULA | |
| Eudragit ® L-100 | 10.0 |
| Triethyl citrate | 2.0 |
| Talc | 1.0 |
| Isopropyl alcohol | q.s. |
| Dichloromethane | q.s. |

Part B: Domperidone film coated tablets (Immediate release)

| | |
|---|---|
| Domperidone | 10.0 |
| Citric acid | 20.0 |
| Vitamin E tocopheryl propylene glycol succinate | 1.0 |
| Lactose | 45.0 |
| Croscarmellose sodium | 10.0 |
| Purified water | q.s. |
| Magnesium stearate | 2.0 |
| Talc | 2.0 |
| COATING FORMULA | |
| Opadry ® white OY-IN-58901 | 2.0 |
| Isopropyl alcohol | q.s. |
| Dichloromethane | q.s. |

Part C: Domperidone enteric coated tablets (Delayed release)

| | |
|---|---|
| Domperidone | 10.0 |
| Citric acid | 20.0 |
| Vitamin E tocopheryl propylene glycol succinate | 1.0 |
| Lactose | 45.0 |
| Croscarmellose sodium | 10.0 |
| Purified water | q.s. |
| Magnesium stearate | 2.0 |
| Talc | 2.0 |
| COATING FORMULA | |
| Opadry ® pink 03B54519 | 2.0 |
| Isopropyl alcohol | q.s. |
| Dichloromethane | q.s. |
| ENTERIC COATING FORMULA | |
| Eudragit ® L-100 | 8.0 |
| Triethyl citrate | 2.0 |
| Talc | 1.0 |
| Isopropyl alcohol | q.s. |
| Dichloromethane | q.s. |

Procedure:

Pantoprazole Tablets
1. Compact Pantoprazole, Anhydrous Sodium carbonate, Magnesium stearate and Talc mixture. Break the compacts.
2. Mix Extra-granular material, Microcrystalline cellulose & Croscarmellose sodium and lubricate with Magnesium stearate and Talc and compress into tablets.
3. Coat the tablets with Opadry® Yellow 03B52544 and then with Eudragit® L-100.

Domperidone Tablets
4. Dissolve Citric acid in hot water. Add Domperidone and Vitamin E TPGS to this hot solution. Stir the slurry for 3-4 hours and allow cooling. Then filter the slurry, dry the residue, and mill to required mesh size.
5. Add Lactose and Croscarmellose sodium, Magnesium stearate and Talc and compress into tablets.
6. Coat half of the tablets with Opadry® white OY-IN-58901.
7. The remaining tablets of step 3 were coated with Opadry® pink 03B54519 and then with Eudragit® L 100.
8. Fill one Pantoprazole tablet, one enteric coated Domperidone tablet and one film coated Domperidone tablet into each hard gelatin capsule.

The Dissolution of the capsule formulated above is carried out using USP Dissolution apparatus Type-2 (paddle) at 100 RPM as follows:

Acid stage: Dissolution medium: 0.1M HCL, 750 ml; Time: 2 hours

Buffer stage: Dissolution medium: Phosphate buffer 6.8 USP, 1000 ml; Time: 1 hour

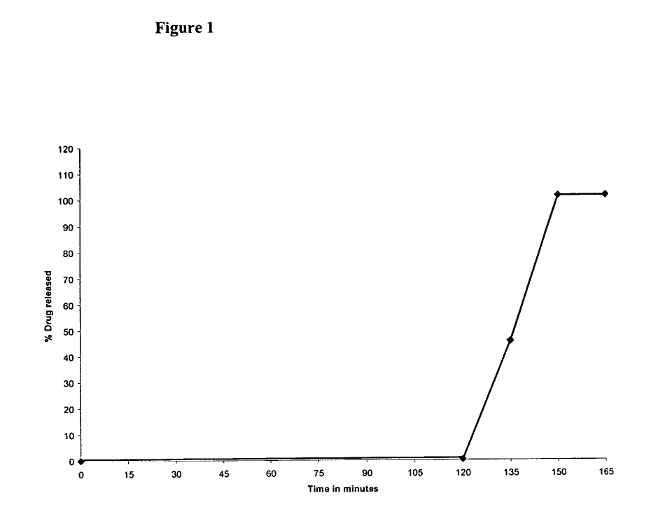

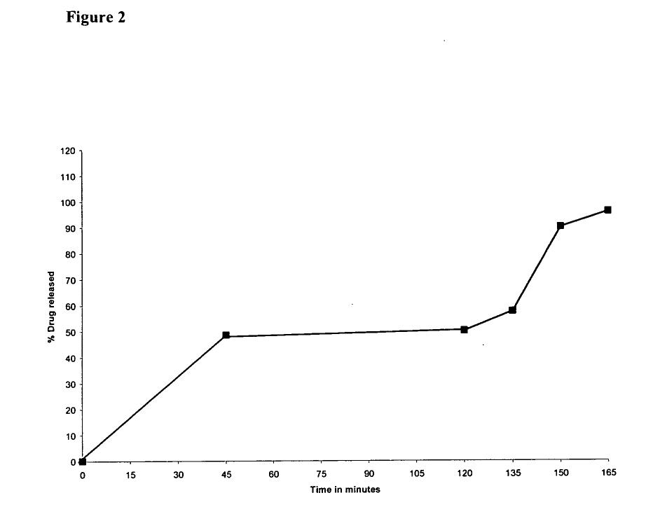

The dissolution profile of Pantoprazole and Domperidone is given below in tables 1 and 2; and is shown in FIGS. 1 and 2 respectively.

TABLE 1
Dissolution profile of Pantoprazole enteric coated tablet

| Dissolution condition | Time (mins.) | % drug released |
|---|---|---|
| | 0 | 0 |
| Acid stage (0.1 N HCl) | 120 | 0.14 |
| Buffer Stage (pH 6.8 phosphate buffer) | 135 | 45.72 |
| Buffer Stage (pH 6.8 phosphate buffer) | 150 | 101.67 |
| Buffer Stage (pH 6.8 phosphate buffer) | 165 | 101.72 |

TABLE 2
Dissolution profile of Domperidone enteric coated tablet

| Dissolution condition | Time (mins.) | % drug released |
|---|---|---|
| Acid stage (0.1 N HCl) | 0 | 0 |
| Acid stage (0.1 N HCl) | 45 | 48.64 |
| Acid stage (0.1 N HCl) | 120 | 50.12 |
| Buffer Stage (pH 6.8 phosphate buffer) | 135 | 57.57 |
| Buffer Stage (pH 6.8 phosphate buffer) | 150 | 89.97 |
| Buffer Stage (pH 6.8 phosphate buffer) | 165 | 96.01 |

### EXAMPLE 2

### Pantoprazole and Metoclopramide Tablets in a Capsule

| | |
|---|---|
| Ingredients | Quantity (mg/Tablet) |

Part A: Pantoprazole Tablets (Delayed release)

| | |
|---|---|
| Pantoprazole sodium sesquihydrate | 45.10 |
| (equivalent to Pantoprazole 40 mg) | |
| Sodium carbonate (anhydrous) | 12.00 |
| Mannitol | 5.00 |
| Microcrystalline cellulose | 5.90 |
| Crospovidone | 15.00 |
| Calcium stearate | 1.00 |
| Talc | 1.00 |
| SEAL COATING FORMULA | |
| Opadry ® yellow 03B52544 | 2.0 |
| Isopropyl alcohol | q.s. |
| Dichloromethane | q.s. |
| ENTERIC COATING FORMULA | |
| Eudragit ® L-100 55 | 10.0 |
| Triethyl citrate | 2.0 |
| Talc | 1.0 |
| Isopropyl alcohol | q.s. |
| Dichloromethane | q.s. |

Part B: Metoclopramide film coated tablets (Immediate release)

| | |
|---|---|
| Metoclopramide | 10.0 |
| Lactose | 45.0 |
| Croscarmellose sodium | 10.0 |
| Magnesium stearate | 2.0 |
| Talc | 2.0 |
| COATING FORMULA | |
| Opadry ® white OY-IN-58901 | 2.0 |
| Isopropyl alcohol | q.s. |
| Dichloromethane | q.s. |

Part C: Metoclopramide enteric coated tablets (Delayed release)

| | |
|---|---|
| Metoclopramide | 20.0 |
| Lactose | 45.0 |
| Croscarmellose sodium | 10.0 |
| Magnesium stearate | 2.0 |
| Talc | 2.0 |
| COATING FORMULA | |
| Opadry ® pink 03B54519 | 2.0 |
| Isopropyl alcohol | q.s. |
| Dichloromethane | q.s. |
| ENTERIC COATING FORMULA | |
| Eudragit ® L-100 55 | 8.0 |
| Triethyl citrate | 2.0 |
| Talc | 1.0 |
| Isopropyl alcohol | q.s. |
| Dichloromethane | q.s. |

Procedure:

Pantoprazole Tablets
1. Slug/deslug Pantoprazole, Anhydrous Sodium carbonate, Calcium stearate and Talc mixture. Break the compacts.
2. Mix Extra-granular material, Mannitol, Microcrystalline cellulose & Crospovidone; lubricate with Calcium stearate and Talc; compress into tablets.
3. Coat the tablets with Opadry® Yellow and then with Eudragit® L-100 55.

Metoclopramide Tablets
4. Mix Metoclopramide, Lactose and Croscarmellose sodium, Magnesium stearate and Talc and compress into tablets.
5. Coat half of the tablets with Opadry® white.
6. The remaining tablets of step 3 were coated with Opadry® pink and then with Eudragit® L-100 55.
7. Fill one Pantoprazole tablet, one enteric coated Metoclopramide tablet and one film coated Metoclopramide tablet into each hard gelatin capsule.

### EXAMPLE 3

### Rabeprazole and Itopride Tablets in a Capsule

Part A: Rabeprazole Tablets (Delayed release)

| Ingredients | Quantity (mg/capsule) |
|---|---|
| Rabeprazole sodium | 20.00 |
| Magnesium oxide (anhydrous) | 80.00 |
| Mannitol | 5.00 |
| Microcrystalline cellulose | 5.90 |
| Crospovidone (Kollidon ® CL) | 15.00 |
| Calcium stearate | 1.00 |
| Talc | 1.00 |
| SEAL COATING FORMULA | |
| Opadry ® yellow 03B52544 | 2.0 |
| Isopropyl alcohol | q.s. |
| Dichloromethane | q.s. |
| ENTERIC COATING FORMULA | |
| Eudragit ® L-100 55 | 10.0 |
| Triethyl citrate | 2.0 |
| Talc | 1.0 |
| Isopropyl alcohol | q.s. |
| Dichloromethane | q.s. |
| Ingredients | Quantity (mg/Tablet) |

Part B: Itopride film coated tablets (Immediate release)

| | |
|---|---|
| Itopride | 50.0 |
| Lactose | 45.0 |
| Croscarmellose sodium | 10.0 |
| Magnesium stearate | 2.0 |
| Talc | 2.0 |
| COATING FORMULA | |
| Opadry ® white OY-IN-58901 | 2.0 |
| Isopropyl alcohol | q.s. |
| Dichloromethane | q.s. |

Part C: Itopride enteric coated tablets (Delayed release)

| | |
|---|---|
| Itopride | 100.0 |
| Lactose | 45.0 |
| Croscarmellose sodium | 10.0 |
| Magnesium stearate | 2.0 |
| Talc | 2.0 |
| COATING FORMULA | |
| Opadry ® pink 03B54519 | 2.0 |
| Isopropyl alcohol | q.s. |
| Dichloromethane | q.s. |
| ENTERIC COATING FORMULA | |
| Eudragit ® L-100 55 | 8.0 |
| Triethyl citrate | 2.0 |
| Talc | 1.0 |
| Isopropyl alcohol | q.s. |
| Dichloromethane | q.s. |

Procedure:

Rabeprazole Tablets
1. Compact Rabeprazole, Anhydrous Magnesium oxide, Calcium stearate and Talc mixture. Break the compacts.
2. Mix Extra-granular material, Mannitol, Microcrystalline cellulose & Kollidon® CL and lubricate with Calcium stearate and Talc and compress into tablets.
3. Coat the tablets with Opadry® Yellow and then with Eudragit® L-100 55.

Itopride Tablets
4. Mix Itopride, Lactose and Croscarmellose sodium, Magnesium stearate and Talc and compress into tablets.
5. Coat half of the tablets with Opadry® white.
6. The remaining tablets of step 3 were coated with Opadry® pink and then with Eudragit® L-100 55.
7. Fill one Rabeprazole tablet, one enteric coated Itopride tablet and one film coated Itopride tablet into each hard gelatin capsule.

### EXAMPLE 4

### Omeprazole and Domperidone Tablets in a Capsule

| | |
|---|---|
| Ingredients | Quantity (mg/Tablet) |

Part A: Omeprazole Tablets (Delayed release)

| | |
|---|---|
| Omeprazole | 40.00 |
| Sodium carbonate (anhydrous) | 10.00 |
| Microcrystalline cellulose | 20.90 |
| Croscarmellose sodium | 20.00 |
| Magnesium stearate | 2.00 |
| Talc | 2.00 |
| SEAL COATING FORMULA | |
| Opadry ® yellow 03B52544 | 2.0 |
| Isopropyl alcohol | q.s. |
| Dichloromethane | q.s. |
| ENTERIC COATING FORMULA | |
| Eudragit ® L-100 | 10.0 |
| Triethyl citrate | 2.0 |
| Talc | 1.0 |
| Isopropyl alcohol | q.s. |
| Dichloromethane | q.s. |

Part B: Domperidone film coated tablets (Immediate release)

| | |
|---|---|
| Domperidone | 10.0 |
| Citric acid | 20.0 |
| Vitamin E tocopheryl propylene glycol succinate | 1.0 |
| Lactose | 45.0 |
| Croscarmellose sodium | 10.0 |
| Purified water | q.s. |
| Magnesium stearate | 2.0 |
| Talc | 2.0 |
| COATING FORMULA | |
| Opadry ® white OY-IN-58901 | 2.0 |
| Isopropyl alcohol | q.s. |
| Dichloromethane | q.s. |

Part C: Domperidone enteric coated tablets (Delayed release)

| | |
|---|---|
| Domperidone | 10.0 |
| Citric acid | 20.0 |
| Vitamin E tocopheryl propylene glycol succinate | 1.0 |
| Lactose | 45.0 |
| Croscarmellose sodium | 10.0 |
| Purified water | q.s. |
| Magnesium stearate | 2.0 |
| Talc | 2.0 |
| COATING FORMULA | |
| Opadry ® pink 03B54519 | 2.0 |
| Isopropyl alcohol | q.s. |
| Dichloromethane | q.s. |
| ENTERIC COATING FORMULA | |
| Eudragit ® L-100 | 8.0 |
| Triethyl citrate | 2.0 |
| Talc | 1.0 |
| Isopropyl alcohol | q.s. |
| Dichloromethane | q.s. |

Procedure:

Omeprazole Tablets
1. Compact Omeprazole, Anhydrous Sodium carbonate, Magnesium stearate and Talc mixture. Break the compacts.
2. Mix extra granular material, Microcrystalline cellulose & Croscarmellose sodium and lubricate with Magnesium stearate and Talc and compress into tablets.
3. Coat the tablets with Opadry® Yellow and then with Eudragit® L-100.

Domperidone Tablets
4. Dissolve citric acid in hot water. Add Domperidone and Vitamin E TPGS to this hot solution. Stir the slurry for 3-4 hours and allow to cool. Then filter the slurry, dry the residue, and mill to required mesh size.
5. Add Lactose and Croscarmellose sodium, Magnesium stearate and Talc and compress into tablets.
6. Coat half of the tablets with Opadry® white.
7. The remaining tablets of step 3 were coated with Opadry® pink and then with Eudragit L 100.

Fill one Pantoprazole tablet, one enteric coated Domperidone tablet and one film coated Domperidone tablet into each hard gelatin capsule.

We claim:

1. An oral pharmaceutical composition comprising at least one gastric acid suppressing agent and one or more prokinetic agent, optionally with other pharmaceutically acceptable excipients, characterized in that the gastric acid suppressing agent is present in a delayed release form and the prokinetic agent is present in a bimodal release form such as an immediate release form to provide an initial loading dose, and a delayed release form to provide a dose with a lag time; with the proviso that the prokinetic agent is not formulated using a rate controlling polymer and is not present in a sustained release form.

2. A composition according to claim 1, wherein the gastric acid suppressing agent is a proton pump inhibitor.

3. A composition according to claim 2, wherein the proton pump inhibitor is selected from the group consisting of pantoprazole, lansoprazole, omeprazole, esomeprazole, rabeprazole, their pharmaceutically acceptable salts, esters, hydrates, or derivatives, used either alone or in combination thereof.

4. A composition according to claim 1, wherein the prokinetic agent is selected from the group consisting of domperidone, metoclopramide, itopride, cisapride, renzapride, zacopride, octreotide, naloxone, erythromycin and bethanechol, Motilides such as Motilin, their pharmaceutically acceptable salts, esters, hydrates, or derivatives, used either alone or in combination thereof.

5. A composition as in any one of claims 1 to 4, wherein the other pharmaceutically acceptable excipients are selected from the group consisting of diluents, binders, disintegrants, colorants, lubricants, plasticizers, coating agents, opacifiers, antioxidants, and the like used either alone or in combination thereof.

6. A composition according to claim 1, which is in the form of tablets filled into hard gelatin capsule.

7. A composition according to claim 1, which is in the form of multilayer tablets.

8. A composition as in any one of claims 1-4 or 6-7, wherein the one prokinetic agent present in the immediate release form and delayed release form is the same.

9. A composition as in any one of claims 1-4 or 6-7, wherein the prokinetic agent present in immediate release form and delayed release form comprises a permeation enhancer.

10. A composition according to claim 5, wherein the one prokinetic agent present in the immediate release form and delayed release form is the same.

11. A composition as claimed in claim 5, wherein the prokinetic agent present in immediate release form and delayed release form comprises a permeation enhancer.

12. A composition as claimed in claim 10, wherein the prokinetic agent present in immediate release form and delayed release form comprises a permeation enhancer.

13. A composition as claimed in claim 9, wherein the permeation enhancer is Vitamin E tocopheryl propylene glycol succinate.

14. A composition as claimed in claim 12, wherein the permeation enhancer is Vitamin E tocopheryl propylene glycol succinate.

15. A process for preparing a composition according to claim 1, comprising at least one gastric acid suppressing agent and one or more prokinetic agent, optionally with other pharmaceutically acceptable excipients, characterized in that the gastric acid suppressing agent is present in a delayed release form and the prokinetic agent is present in a bimodal release form such as an immediate release form to provide an initial loading dose, and a delayed release form to provide a dose with a lag time; with the proviso that the prokinetic agent is not formulated using a hydrophilic rate controlling polymer and is not present in a sustained release form, which comprises of the steps:
i) processing the acid suppressing agent with pharmaceutically acceptable excipients,
ii) processing the prokinetic agent with pharmaceutically acceptable excipients,
iii) formulating the material of step i) and ii) into a suitable dosage form.

16. A process for preparing a composition according to claim 15, comprising at least one gastric acid suppressing agent and one or more prokinetic agent, optionally with other pharmaceutically acceptable excipients, characterized in that the gastric acid suppressing agent is present in a delayed release form and the prokinetic agent is present in a bimodal release form such as an immediate release form to provide an initial loading dose, and a delayed release form to provide a dose with a lag time; with the proviso that the prokinetic agent is not formulated using a hydrophilic rate controlling polymer and is not present in a sustained release form, which comprises of the steps:
i) processing the acid suppressing agent with pharmaceutically acceptable excipients into enteric coated tablets,
ii) processing the prokinetic agent with pharmaceutically acceptable excipients partly into film coated tablets and partly into enteric coated tablets,
iii) filling one enteric coated tablet comprising an acid suppressing agent, and one film coated tablet and one enteric coated tablet comprising a prokinetic agent, into a hard gelatin capsule.

17. A process for preparing a composition according to claim 15, comprising at least one gastric acid suppressing agent and one or more prokinetic agent, optionally with other pharmaceutically acceptable excipients, characterized in that the gastric acid suppressing agent is present in a delayed release form and the prokinetic agent is present in a bimodal release form such as an immediate release form to provide an initial loading dose, and a delayed release form to provide a dose with a lag time; with the proviso that the prokinetic agent is not formulated using a hydrophilic rate controlling polymer and is not present in a sustained release form, which comprises of the steps:
i) processing the acid suppressing agent with pharmaceutically acceptable excipients into enteric granules,
ii) processing one part of the prokinetic agent with pharmaceutically acceptable excipients into immediate release granules, and the other part into enteric granules,
iii) compressing the granules of step i) and ii) into a multilayer tablet and
iv) optionally coating the tablet.

18. A method of treatment of gastro esophageal reflux disease, reflux esophagitis, peptic ulcer, gastric ulcer, and other gastric acid related disorders by administering to a patient in need thereof a pharmaceutical composition according to claim 1.
